(19) United States
(12) Patent Application Publication
(10) Pub. No.: US 2024/0238104 A1
(43) Pub. Date: Jul. 18, 2024

(54) SYSTEM AND METHOD FOR MAKING A BREAST PROSTHESIS

(71) Applicant: EVE BORSTPROTHESES B.V., Haarlem (NL)

(72) Inventor: Norman Martin VAN BEEK, Driehuis (NL)

(21) Appl. No.: 18/562,112

(22) PCT Filed: May 27, 2022
(86) PCT No.: PCT/EP2022/064394

§ 371 Date: Nov. 17, 2023

(30) Foreign Application Priority Data
May 28, 2021 (NL) 2028328

(51) Int. Cl. A61F 2/52; A61F 2/50; B29C 45/00; B29C 45/34; B29C 51/10; B29C 64/188; B29C 64/386; B29K 83/00; B29L 31/00; B33Y 40/20; B33Y 50/00; B33Y 80/00

(52) U.S. Cl. CPC A61F 2/52; B29C 45/0053; B29C 45/34; B29C 51/10; B29C 64/188; B29C 64/386; B33Y 40/20; B33Y 50/00; B33Y 80/00; A61F 2002/501; A61F 2002/505; A61F 2002/5053; A61F 2002/5056; B29K 2083/00; B29K 2863/00; B29K 2867/003; B29L 2031/7532

(57) ABSTRACT

Mould assembly for injection moulding a personalised hollow breast prosthesis, wherein the mould assembly comprises: —a front mould being a female mould of a front side of a patients breast and comprising a first mould surface, —a rear mould being a female mould of a patients mastectomized chest and comprising a second mould surface, —a mould core comprising a mould core main body being a scaled positive mould of a combination of the patients breast and the patients mastectomized chest, wherein the mould core is smaller than the combination, —a support configured to support the mould core main body in an assembled state of the mould assembly, wherein an injection opening is defined by any of the front mould, the rear mould, and the mould core, and wherein in the assembled state the mould core is located between the front mould and the rear mould in order for a mould core surface to be located at a distance from the first mould surface and the second mould surface, wherein in the assembled state an inner volume is defined by the front mould, the rear mould and mould core and is configured to be filled with an injection moulding material.

### FIELD OF THE INVENTION

The field of the invention relates to the manufacturing of personalized breast prosthesis.

### BACKGROUND OF THE INVENTION

The manufacturing of personalized breast prosthesis in itself is known. Currently, methods exist to create personalized breast prosthesis that are created with the aid of additive manufacturing.

CN105877879A discloses a method of producing a personalized breast prosthesis wherein a three dimensional model is created using MRI and CT scans. The model that is based on these scans is then printed to form a mould. Subsequently, the mould is filled with a material that is suitable for the creation of a prosthesis, e.g. silica gel, and is solidified to form a personalized breast prosthesis.

It was recognized in the present invention that a potential drawback of the described method of CN105877879A is that the resulting prosthesis is relatively heavy because the mould must be entirely filled up to a level that the prosthesis is sufficiently large. The resulting prosthesis will also be very firm and will generally not provide a realistic feel.

Also, because the mould is filled and a free surface of material is created in the process, the rear of the prosthesis will be flat and can be uncomfortable for a user to wear.

CN110025413A relates to a method for manufacturing a personalized breast prosthesis. The method comprises the step of scanning a post-operative breast, i.e. the mastectomized chest, of an individual. During the establishment of a digital model of the breast prosthesis, a scan of a healthy breast and of the mastectomized chest are combined to form a model of the breast prosthesis. Thereafter, a female and a male mould are printed and trimmed or modified. Subsequently, after having assembled the moulds, raw material is injected into the mould and is cured.

Even though the prosthesis is configured to fit on the mastectomized chest of a user, it still has several drawbacks. Because the mould must be entirely filled with the raw material, the resulting prosthesis may be very firm and heavy. This is believed not to contribute to a realistic feel of the prosthesis.

Also, because the moulds are 3D-printed, the resolution of the printing will play a major role in the surface finish of the prosthesis; a lot of time will have to be spent on printing with a very high resolution or on a finishing step of the prosthesis, i.e. sanding or trimming.

NL7712084A relates to a breast prosthesis and a method for the manufacturing of the breast prosthesis. A mould is provided with comprises an upper part, a lower part, and an interchangeable core. For the manufacturing of a prosthesis, a prefabricated latex bag is placed partially over the core and a ribbed body is slid over the core. The ribbed body engages the latex bag at a groove. Subsequently, the air present in the latex bag is evacuated through a channel extending through the core and a two-component polymer is injected into the bag. Thereafter, the core is removed and the ribbed body is removed.

The ribbed body is necessary to avoid creating pressure points on a mastectomized chest of an individual. Therefore, a trade-off has to be made between comfort and realistic feel. Even though the prosthesis may be comfortable to wear, a non-uniform wall-thickness potentially causes a non-realistic feel of the prosthesis.

Further, the use of a prefabricated latex bag only makes it possible to manufacture a single size prosthesis; if someone desires a specific size of prosthesis, a specific size of latex bag will have to be found. Additionally, if a shape of the prosthesis would be modified, the latex bag would also have to be modified to avoid wrinkling, folding, or stretching of the bag.

Also, because air cannot escape through the latex bag, the evacuation of air from the bag is necessary. This may potentially damage the latex bag.

### OBJECT OF THE INVENTION

The object of the invention is to overcome at least of the abovementioned drawbacks, and in particular to provide a device and a method for the production of a personalized breast prosthesis that matches a patient's chest.

### SUMMARY OF THE INVENTION

The invention relates to a mould assembly for injection moulding a personalised hollow breast prosthesis, wherein the mould assembly comprises:
a front mould being a female mould of a front side of a patients breast and comprising a first mould surface,
a rear mould being a female mould of a patients mastectomized chest and comprising a second mould surface,
a mould core comprising a mould core main body being a scaled positive mould of a combination of the patients breast and the patients mastectomized chest, wherein the mould core is smaller than the combination,
a support configured to support the mould core main body in an assembled state of the mould assembly and to hold the mould core main body in a predetermined position relative to the front mould and rear mould,

wherein an injection opening is defined by any of the front mould, the rear mould, and the mould core, and wherein in the assembled state the mould core is located between the front mould and the rear mould in order for a mould core surface to be located at a distance from the first mould surface and the second mould surface, wherein in the assembled state an inner volume is defined by the front mould, the rear mould and the mould core, wherein the inner volume extends around the mould core and is configured to be filled with an injection moulding material.

By using a mould assembly as described above, a personalized prosthesis can be efficiently be created for an individual patient. The prosthesis will not only match the individuals original breast from an external point of view, it will also comprise a rear surface that substantially matched the mastectomized chest of the individual. After a surgery, it is beneficial to the healing of a wound area to exert a pressure on the wound area. If a non-personalized prosthesis would be used, pressure points would be created on the individuals chest causing discomfort and less than optimal healing. The rear side of the breast prosthesis manufactured using abovementioned mould assembly matches the wound area and therefore distributes an even pressure, reducing those disadvantages.

Also, by using a supported mould core, a hollow prosthesis can be created. This may enhance the realistic feel of the prosthesis and may also reduce the weight of the prosthesis.

In an embodiment, the support and the mould core are integral and the support protrudes outwards from a rear side of a mould core main body and is configured to engage the rear mould, wherein the support has a smaller cross-section than the mould core main body.

By having a separate mould core, after a prosthesis has been injection moulded, the prosthesis and the mould core can be taken out of the mould assembly. This facilitates the handling and removal of the mould core from the prosthesis. Because the support protrudes outwards from the mould core body, it offers a handle to exert forces on and manipulate the mould core.

In an embodiment, the rear mould defines a support hole, wherein in the assembled state the support extends into the support hole, in particular the support hole being a through hole wherein the support extends into the support hole and is flush with a rear side of the rear mould.

By placing the support in a support hole, the mould core can be accurately positioned and kept in a specific position.

In another embodiment, the support is part of the rear mould and protrudes away from the second mould surface and towards the mould core and is configured to engage the mould core, wherein the second mould surface is adjacent to the inner volume.

When the support is part of the rear mould instead of the mould core, the mould core may be more easily manufacturable.

In an embodiment, the mould core defines a support hole, wherein in the assembled state the support extends into the support hole.

By placing the support in a support hole, the mould core can be accurately positioned and kept in a specific position.

In an embodiment, the support and the support hole have a substantially equal, non-circular cross-section.

The substantially equal cross-section limits the support and support hole to translate in a lateral direction or to rotate about a lateral axis relative to each other. The non-circularity of the cross-section prevents the support from rotating about an upwardly oriented axis relative to the support hole. In doing so, the accuracy of the placement of the mould core is enhanced.

In another embodiment, the support extends between the mould core and the rear mould and wherein the support, the mould core and the rear mould are an integral part. Alternatively, the support extends between the mould core and the front mould and wherein the support, the mould core and the front mould are an integral part.

In doing so, relative movements between the mould core and the rear mould or between the mould core and the front mould are reduced. In doing so, the accuracy of the placement of the mould core is enhanced.

In an embodiment, a circumference of the support is at least 30 percent of the largest dimension of the mould core.

Because the mould core has to be removed from a breast prosthesis that has been injection moulded using the mould assembly, a hole must be present in the resulting prosthesis. Subsequently, because the support extends between the mould core and the rear mould or the front mould, a hole is created in the prosthesis during the injection moulding at the location where the support is located. The circumference of the support therefore dictates the circumference of the hole through which the mould core must be removed. Therefore the hole must be 30 percent of the largest dimension of the mould core to be able to remove the mould core.

In an embodiment, the distance from the mould core surface to the first mould surface and the second mould surface is substantially uniform over the entire mould core surface, in particular the distance being in the range of 3-8 mm, more in particular the distance being 5 mm.

In doing so, the wall thickness of an injection moulded prosthesis may also be substantially uniform. This can contribute to the realistic feel of the prosthesis and its durability.

In an embodiment, the injection opening is located at a front side of the inner volume and is configured to allow injection of a polymer near the front mould.

When the injection opening is located at the front side of the inner volume, a polymer may be injected at the front of what is to become the prosthesis. In doing so, because the polymer will flow around the mould core, the joining of the different flow directions will occur on a back side of the inner volume. Any flow markings will then also be located on the rear side where they are not visible when the prosthesis is worn.

In an embodiment, an injection channel extends through the support and through the mould core main body and to the injection opening and is configured to allow injection of a polymer from outside the mould assembly into the mould assembly. In particular the injection channel has a diameter of 3-15 mm, in particular 5-11 mm, more in particular 7-9 mm.

By allowing the injected polymer to enter the inner volume from within, i.e. not through the front mould or the rear mould, markings and excess material on the resulting prosthesis from the injection moulding process will also be located within the prosthesis and will not be visible from the outside. Either this enhances the appearance of the prosthesis or reduces the finishing steps to remove markings and excess material.

In an embodiment, the front mould is placed over the rear mould in the assembled state, or vice versa, and wherein at least one of the front mould and the rear mould defines at least one air channel being configured to allow air to escape when the mould assembly is being filled, in particular the at least one air channel having a diameter of 0.5-3 mm, in particular 1-2 mm.

Prior to filling the mould assembly, the inner volume will be filled with air. When the material is injected into the mould, the air has to be removed from the inner volume. The at least one air channel allows the egress of air during the filling process.

In an embodiment, the cross-section of the support is smaller than a cross-section of the support hole and wherein, in the assembled state, an air channel is defined between the support and the support hole.

By locally leaving room between the support and the support hole, an air channel is created through which air from the inner volume may be expelled.

In an embodiment, in the assembled state, the front mould is fixed to the rear mould by fixating means, form-fit and/or force-fit, and/or wherein the mould core is fixed to the rear mould by fixating means, form-fit and/or force-fit.

The front and rear mould should be fixed with respect to each other to reduce injected material flowing out of the mould assembly and to increase alignment accuracy.

In an embodiment, the front mould comprises a skirt and the rear mould comprises a wall, or vice versa, wherein in the assembled state, the skirt is form-fitted to the wall.

The use of a skirt and a wall results in an accurate placement of the front mould relative to the rear mould.

In an embodiment, the skirt and the wall are oriented substantially upwards in the assembled state. This may improve the flow of the injected material.

In an embodiment, the wall defines an end stop, wherein the skirt abuts on the end stop in the assembled state, wherein the end stop is configured to ensure an accurate positioning of the skirt in an X-direction, a Y-direction and a Z-direction.

In an embodiment, the rear mould comprises a separate first part and a separate second part and the separate first part and the separate second part are connected to each other via a bridge section. This bridge section may be configured to be broken after having injected a polymer in the mould assembly to easily separate the moulds. In particular this aids in the removal of the mould core from the rear mould.

In an embodiment, any of the front mould, rear mould, and mould core are manufactured using an additive manufacturing method, in particular 3D-printing.

In an embodiment, the front mould has been manufactured via vacuum forming. The process of vacuum forming may create a very smooth front surface of a resulting prosthesis.

In another aspect, the invention relates to a method for injection moulding a personalized hollow breast prosthesis using a mould assembly, wherein the method comprises the steps:
a) obtaining a first 3D-scan of a breast of a patient by 3D-scanning the patient,
b) obtaining a second 3D-scan of a mastectomized chest of a patient by 3D-scanning the patient,
c) calculating a 3D-model of the breast based on the first 3D-scan and calculating a 3D-model of the mastectomized chest based on the second 3D-scan,
d) creating a front mould model, a rear mould model, and a mould core model based on the 3D-model of the breast and the 3D-model of the mastectomized chest,
e) 1) 3D-printing an intermediary front mould corresponding to the front mould model and creating a front mould from the intermediary front mould, 3D-printing a rear mould corresponding to the rear mould model, and 3D-printing a mould core corresponding to the mould core model, or
   2) 3D-printing a front mould corresponding to the front mould model, 3D-printing a rear mould corresponding to the rear mould model, and 3D-printing a mould core corresponding to the mould core model,
f) assembling the front mould, the rear mould, and the mould core into the mould assembly,
g) injecting a polymer into the mould assembly through an injection opening creating a hollow breast prosthesis,
   wherein in the assembled state a mould core main body is supported by a support to position a mould core surface at a distance from the front mould and the rear mould.

By obtaining 3D-scans of a patients breast and mastectomized chest, a prosthesis can be manufactured that largely corresponds to a patient's body. The front of the prosthesis will look substantially like the patient's original breast and the rear of the prosthesis will be suited to fit the mastectomized chest of a patient. Further, by creating a three-piece mould assembly, a hollow prosthesis can be created. This may enhance the realistic feel of the prosthesis and may also reduce the weight of the prosthesis.

By using 3D-printing and 3D-scanning to create the mould assembly, a personalized prosthesis can be created in relatively little time and for relatively low cost. In doing so, such a personalized prosthesis may become accessible to a large number of people in need of a prosthesis.

In an embodiment, in step e1), the front mould is created by vacuum forming a mould material onto the intermediary front mould, in particular the mould material being PET.

By vacuum forming, a mould with a smooth surface can be obtained relatively easy an may reduce production time, because a print resolution can be relatively low and less finishing steps may have to be taken.

In an embodiment, the intermediary front mould is a positive vacuum forming mould. Herein, the mould material would be vacuum formed over the intermediary front mould. A benefit of such an intermediary front mould is the manufacturing of the intermediary front mould takes relatively little time.

In an embodiment, after step e) at least one of the front mould, the intermediary front mould, the rear mould, and the mould core are covered with a filler material to reduce surface imperfections, in particular the filler material being epoxy.

By reducing surface imperfections on at least one of the front mould, the intermediary front mould, the rear mould, and the mould core, a smoother prosthesis may be obtained.

In an embodiment, the distance from the mould core surface to a first mould surface and to a second mould surface is substantially uniform over the entire surface of the mould core and during step g) the mould core is partially surrounded by the polymer to form a hollow breast prosthesis, in particular the distance being in the range of 3-8 mm, more in particular the distance being 5 mm. This can contribute to the realistic feel of the prosthesis and its durability.

In an embodiment, the support is part of the mould core and protrudes outwards from a rear side of a mould core body and engages the rear mould during step f). Alternatively, the support is part of the rear mould and protrudes away from a second mould surface of the rear mould and engages the mould core during step f).

By having a separate mould core, after step g), the prosthesis and the mould core can easily be taken out of the mould assembly. This facilitates the handling and removal of the mould core from the prosthesis. Because the support protrudes outwards from the mould core body, it offers a handle to exert forces on the mould core.

When the support is part of the rear mould instead of the mould core, the mould core may be more easily manufacturable.

In an embodiment, the rear mould defines a support hole, in particular a through hole, wherein during step f) the support is placed in the support hole. Alternatively, the mould core defines a support hole, wherein during step f) the support is placed in the support hole.

By placing the support in a support hole, the mould core can be accurately positioned and kept in a specific position.

In an embodiment, the support extends between the mould core and the rear mould and the support, the mould core and the rear mould are an integral part. Alternatively, wherein the support extends between the mould core and the front mould and wherein the support, the mould core and the front mould are an integral part.

In doing so, relative movements between the mould core and the rear mould or between the mould core and the front mould are reduced. In doing so, the accuracy of the placement of the mould core is enhanced.

In an embodiment, the mould core defines an injection channel extending to the injection opening and during step g) the polymer flows through the injection channel of the mould core into the mould assembly.

By allowing the injected polymer to enter the inner volume from within, i.e. not through the front mould or the rear mould, markings and excess material on the resulting prosthesis from the injection moulding process will also be located within the prosthesis and will not be visible from the outside. This enhances the appearance of the prosthesis and reduces the finishing steps to remove markings and excess material.

In an embodiment, between step f) and step g) the mould assembly is rotated to an orientation wherein the rear mould is located above the mould core.

By orienting the mould assembly in the above manner, a better flow path may be created for the injected material.

In an embodiment, after step g) the mould assembly is disassembled and the mould core is removed from the hollow breast prosthesis.

In an embodiment, after step g) the mould core is removed from the hollow breast prosthesis through a hole in the hollow breast prosthesis created by the support during step g).

In an embodiment, the hollow breast prosthesis is stretched to allow the removal of the mould core through the hole in the hollow breast prosthesis, and wherein in particular an edge which surrounds, and in particular defines, the hole is stretched to remove the mould core through the hole.

In an embodiment, the rear mould comprises a separate first part, a separate second part, and a bridge section connecting the separate first part and the separate second part that is configured to be broken after step g). During step e) the separate first part, the separate second part and the bridge section are 3D-printed into an integral part.

In another embodiment, the rear mould comprises a separate first part, a separate second part, and a bridge section connecting the separate first part and the separate second part that is configured to be broken after step g). During step e) the separate first part and the separate second part are 3D-printed individually and are thereafter joined together by the bridge section. Herein, the bridge section is created by melting and solidifying a polymer between the separate first part and the separate second part.

In an embodiment, after step g) the bridge section is broken and the separate first part is separated from the separate second part.

By creating a bridge section that is configured to be broken after step g) and breaking it after step g), it is possible to unmould the breast prosthesis more easily than with a solid rear mould.

In an embodiment, the hole in the hollow breast prosthesis is closed with a seal after removing the mould core.

By closing the hole with a seal, the medium inside the hollow prosthesis cannot escape the prosthesis. This reduces the compressibility of the prosthesis and contributes to a realistic feel.

In an embodiment, the hollow breast prosthesis comprises a valve and the hollow breast prosthesis is filled with a medium or is drained of a medium through the valve, in particular the valve being located in the seal, more in particular the medium being air.

By utilizing a valve for the filling or draining of the prosthesis, compressibility of the prosthesis may be adjusted. Not only does this contribute to a desired feel of the prosthesis, it may also be used to exert a specific pressure on a patient's chest when the prosthesis is used.

In an embodiment, edges of the breast prosthesis are trimmed after step g).

In an embodiment, after step g) a least one adhering patch is fixed to the rear of the breast prosthesis, wherein the at least one adhering patch is configured to adhere to a patient's chest. By fixing at least one adhering patch to the rear of the breast prosthesis, it may be kept in place on the patient's body.

In an embodiment, the at least one patch is fixed to the breast prosthesis with liquid silicone that has been hardened.

In an embodiment, the adhering patch comprises a nano-structure that interacts with a patient's skin to adhere to the patient's chest, in particular the nano-structure being created by pouring silicone on a diffraction grating. By using a patch with a nano-structure, the prosthesis can be fixated without glue. This means that the prosthesis can be placed and removed time after time without have to apply new glue and without having to remove glue residue from the patient's body. Diffraction grating has been found to have the desired characteristics and structure to be suitable for this purpose.

In an embodiment, the first 3D-scan is obtained from a breast that is not to be mastectomized and prior to step d) the first 3D-scan or the 3D-model of the breast based on the first 3D-scan is mirrored.

In doing so, a symmetrical chest can be obtained when using the resulting prosthesis. Additionally, the personalized prosthesis may also be created after a mastectomy.

In an embodiment, prior to step f) errors and/or imperfections are removed from at least one of the first 3D-scan, the second 3D-scan, the 3D-model of the breast, the 3D-model of the mastectomized chest, the front mould model, the rear mould model, the mould core model, in particular by computer editing, and from at least one of the intermediary front mould, the front mould, the rear mould, and the mould core, in particular by sanding.

In an embodiment, during step c) a single 3D-model is calculated based on both the first 3D-scan and the second 3D-scan, and the model is split into a breast part and a rear part during step d), and the front mould model is based on the breast part, the rear mould model is based on the rear part, and the mould core model is based on the single model.

In an embodiment, the polymer is silicone.

In an embodiment, a polymer colour is determined from the first scan and/or the second scan prior to step g). This may contribute to a final appearance of the prosthesis and to match the prosthesis to a patient. The colour may also be determined by hand using colour swatches.

In an embodiment, the polymer is hardened after step g), in particular cured.

In an embodiment, step g) is performed while the mould assembly is oriented in an injection orientation, wherein the front mould is located above the rear mould.

By orienting the mould assembly in the above manner, a better flow path may be created for the injected material.

In an embodiment, the first 3D-scan and the second 3D-scan are made using a 3D-scanner.

In an embodiment, the mould assembly is a mould assembly according to any of claims 1-19.

In a further aspect, the invention relates to a personalized hollow breast prosthesis, comprising:
a hollow shell defining a hole, wherein the hole is located on a rear side of the shell,
a seal being located at the hole, wherein the seal is fixed to the hollow shell and seals the hole,
   wherein an inner volume is defined by the hollow shell and the seal, the inner volume being filled with a medium, in particular the medium being air.

Such a prosthesis may have a relatively low weight because of its hollow character and may feel relatively authentic due to the trapped medium inside the inner volume.

In an embodiment, the seal or the hollow shell comprises a valve, wherein the personalized hollow breast prosthesis is configured to be inflated or deflated via the valve.

By utilizing a valve for the filling or draining of the prosthesis, compressibility of the prosthesis may be adjusted. Not only does this contribute to a desired feel of the prosthesis, it may also be used to exert a specific pressure on a patient's chest when the prosthesis is used.

In an embodiment, the shell comprises a wall and the wall has a substantially uniform thickness. Not only can a uniform wall thickness contribute to the feel of the prosthesis, is may also be beneficial to its durability.

In an embodiment, the shell and/or the seal are made of silicone.

In an embodiment, a front part and a rear part of the prosthesis are a single integral part.

In an embodiment, at least one adhering patch is fixed to the rear of the breast prosthesis, wherein the at least one adhering patch is configured to adhere to a patient's chest. By fixing an adhering patch to the rear of the breast prosthesis, it may be kept in place on the patient's body.

In an embodiment, the patch is connected to the breast prosthesis with hardened liquid silicone.

In an embodiment, one adhering patch substantially covers the rear of the breast prosthesis and substantially matches a rear surface of the breast prosthesis. In doing so, an optimal adhering property can be achieved because there is a large amount of surface interaction.

In an embodiment, a plurality of adhering patches covers part of the rear of the breast prosthesis. In particular, the patches can be circular and/or elongated in form.

This reduces the amount of skin that is directly covered by the prosthesis while still keeping it in place. This may increase the wearing comfort for the user.

In an embodiment, the at least one adhering patch comprises a nano-structure that interacts with a patient's skin to adhere to the patient's chest.

In an embodiment, the at least one adhering patch has been manufactured by pouring liquid silicone on a diffraction grating and letting it harden. In particular, the nano-structure that is formed is a negative of the diffraction grating.

By using a patch with a nano-structure, the prosthesis can be fixated without glue. This means that the prosthesis can be placed and removed time after time without have to apply new glue and without having to remove glue residue from the patient's body. Diffraction grating has been found to have the desired characteristics and structure to be suitable for this purpose.

In an embodiment, the prosthesis has been made using the method according to any of claims 20-43 and/or with the mould assembly according to any of claims 1-19.

The invention will be more clearly understood from the following description of some preferred embodiments, which are given by way of example only, with reference to the accompanying drawings.

### BRIEF DESCRIPTION OF THE DRAWINGS

FIG. 1 shows a cross-section in side view of an embodiment of the mould assembly.

FIG. 2 shows a cross-section in side view of an exploded view of an embodiment of the mould assembly.

FIG. 3 shows a cross-section in side view of an embodiment of the mould assembly.

FIGS. 4A, 4B, and 4C show isometric views of the intermediary front mould and the front mould.

FIGS. 5A and 5B show a side view and a front view of the rear mould.

FIGS. 6A and 6B show a side view and a front view of the mould core.

FIGS. 7A-7D show side views of the mould core and the breast prosthesis.

FIGS. 8A-8D show side and front views of a patient's chest with and without the breast prosthesis.

FIGS. 9A, 9B, and 9C show the personalized hollow breast prosthesis with and without the seal.

FIGS. 10A and 10B show the adhering patch on the breast prosthesis.

### DETAILED DESCRIPTION OF THE DRAWINGS

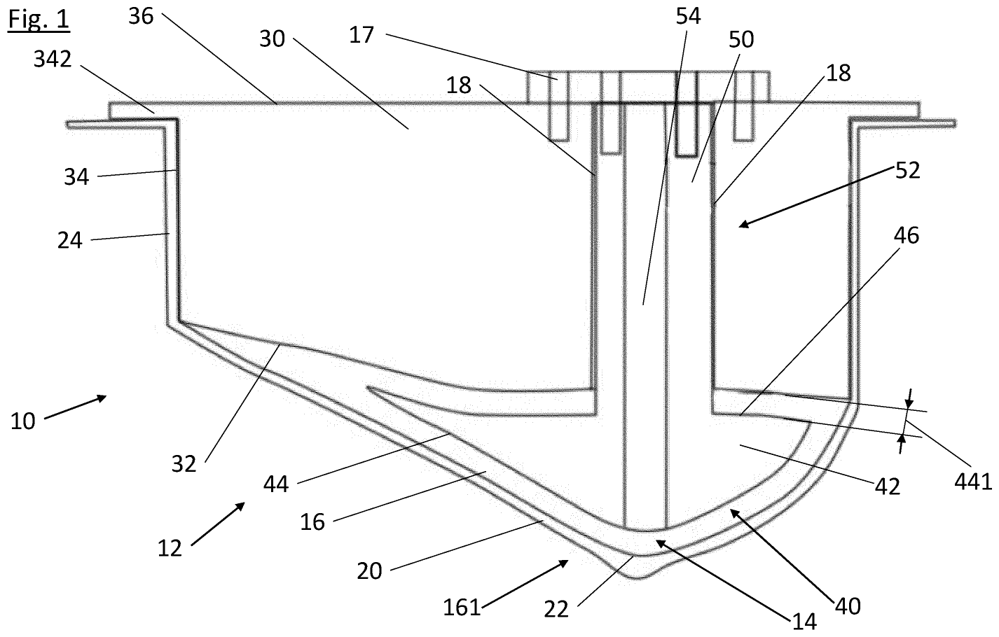

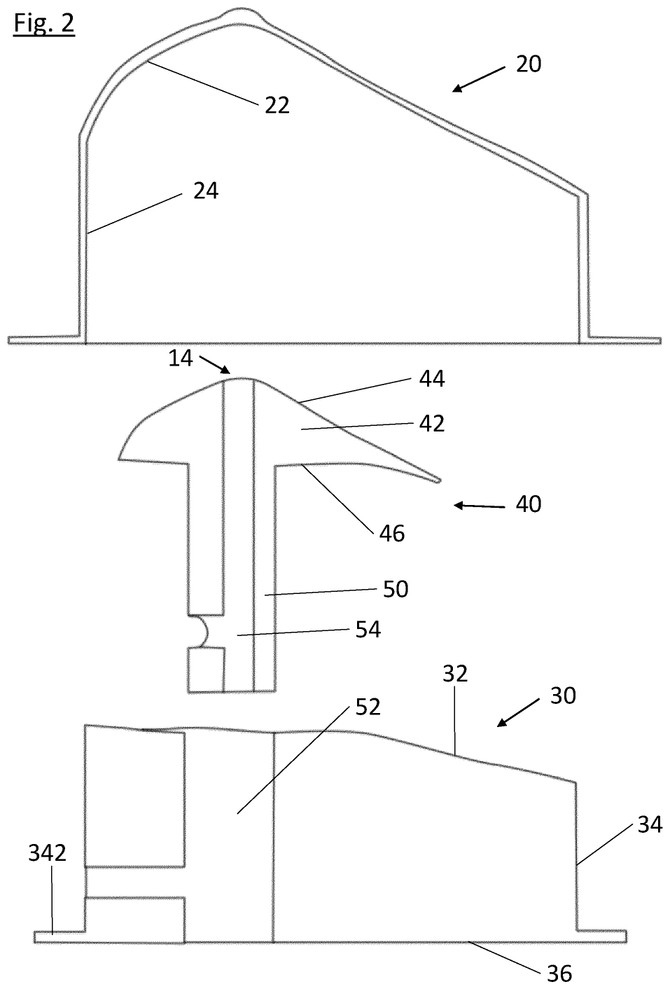

In FIGS. 1 and 2, a cross-section of a mould assembly 10 for injection moulding a personalized hollow breast prosthesis is shown. In FIG. 1, the mould assembly 10 is shown in an assembled state 12 and in FIG. 2, is it shown in an exploded view. To arrive at the assembled state 12, a mould core 40 engages a rear mould 30 and a front mould is placed over the rear mould 30.

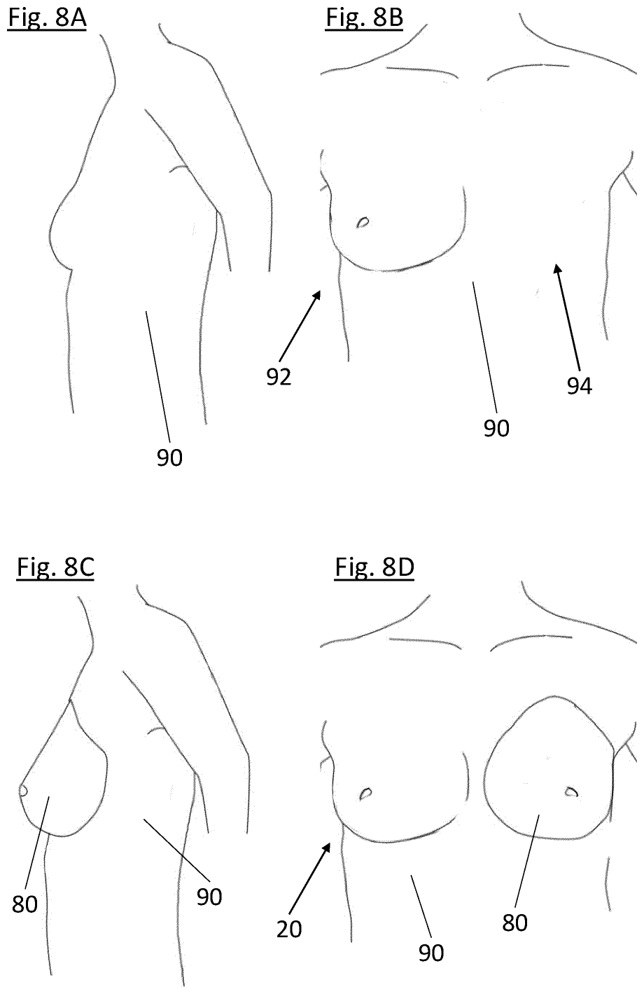

The mould assembly comprises a front mould 20 that is a negative of a patients breast 92 (depicted in FIG. 8B) and comprises a first mould surface 22. It is this surface that will create a front exterior side of a breast prosthesis manufactured with the mould assembly.

A rear mould 30 of the mould assembly is a negative of a patients mastectomized chest 94 (depicted in FIG. 8B). The rear mould 30 comprises a second mould surface 32 that will create a rear exterior side of the breast prosthesis manufactured with the mould assembly.

In order to be able to create a hollow breast prothesis, a mould core 40 is provided in between the front mould 20 and the rear mould 30. The mould core comprises a mould core main body 42 that is a scaled down positive mould of a combination of the patients breast 92 and the patient's mastectomized chest 94 and that is smaller than the combination. The mould core will create an interior side of the breast prosthesis manufactured with the mould assembly.

For a mould core surface 44 of the mould core 40 to be located between the front mould 20 and the rear mould 30 and at a distance 441 from the first mould surface 22 and the second mould surface 32, a support 50 is provided that supports the mould core main body in the assembled state. The presence of a distance between the mould core and the front and rear mould, creates an inner volume 16 into which an injection material can be injected. To be able to do so, the mould core defines an injection opening 14 through which the material can enter the mould assembly. This injection opening 14 is located at a front side 161 of the inner volume to allow injection of a polymer near the front mould to create a desirable flow path.

Further, to achieve a desirable prosthesis feeling, it may be beneficial that the wall thickness of the breast prosthesis is substantially uniform; to this end the distance 441 is substantially uniform over the entire mould core surface 44. Also, when the injection moulding material is injected into the mould assembly 10, air located in the inner volume 16 must be evacuated. To this end, an air channel 18 is present between the support and the rear mould, wherein the cross-section of the support 50 is slightly smaller than a cross-section of the support hole 52 and the air channel is defined between the two.

Here, the support 50 is part of the mould core 40 and protrudes outwards from a rear side 46 of the mould core main body 42. The rear mould 30, in turn, defines a support hole 52 that is a through hole being configured to accommodate the support 50. The support 50 (having a smaller cross-section than the mould core main body 42) is configured to engage the rear mould 30 by being placed in the support hole 52 where it is flush with a rear side 36 of the rear mould and is kept in place by fixating means 17 in the form of bolts.

Because, the injection opening 14 is located within the mould assembly, an injection channel 54 extends through the mould core main body 42 and through the support 50 to allow injection of a polymer from outside the mould assembly into the mould assembly. To allow a useful flow, the injection channel 54 has a diameter of 3-15 mm, in particular 5-11 mm, more in particular 7-9 mm.

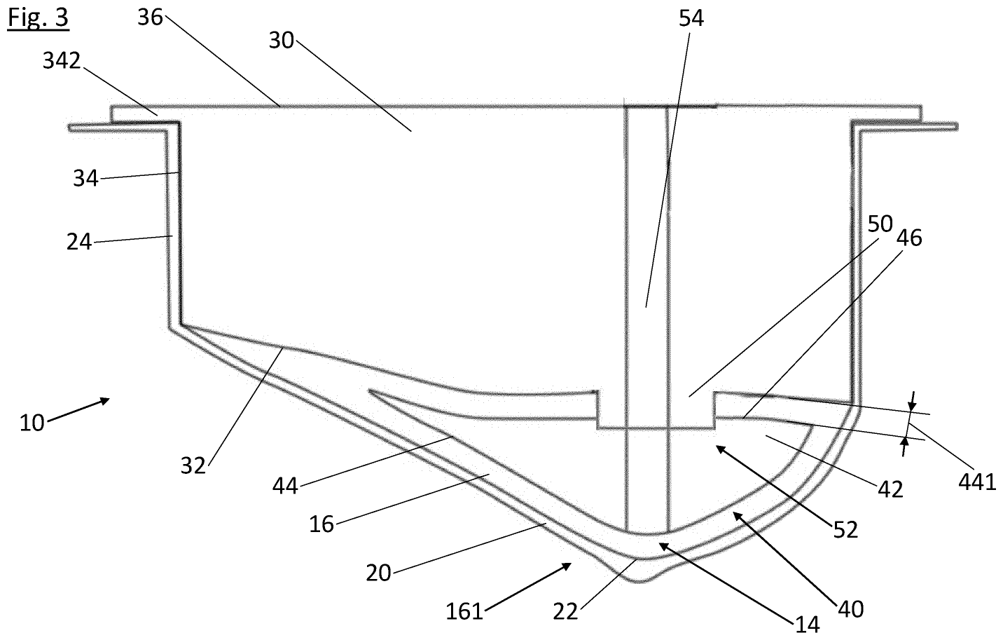

Turning to FIG. 3, a similar mould assembly 10 as mentioned above is shown. However, the support 50 is now part of the rear mould and protrudes away from the second mould surface 32 instead of being part of the mould core 40. The support 50 is now configured to engage the mould core 40 via a support hole 52, defined by the mould core 40, by extending into the support hole 52. In order for the mould core 40 not to rotate between the front mould 20 and the rear mould 30, the cross-sections of the support and the support hole have a substantially equal, non-circular cross-section. Here, instead of being fixed by fixating means 17 such as bolts or screws, the mould core is form-fit to the rear mould.

In order for the injected material not to leak out of the mould assembly 10, the front mould 20 comprises a skirt 24 that is form fitted to a wall 34 of the rear mould 30. Also, to ensure accurate positioning of the skirt in all directions, the wall 34 comprises an end-stop 342 upon which the skirt 24 abuts in the assembled state.

Other constructions, such as a support extending between the mould core and the rear mould or the front mould and being an integral part with the rear or front mould may also be realized. However, in order to be able to remove the hollow breast prosthesis from the mould core, a circumference of the support is at least 30 percent of the largest dimensions of the mould core.

To be able to injection mould a personalized breast prosthesis, the mould assembly 10 should be made specifically for a specific patient. Therefore, in order to arrive at a mould assembly that is suitable for this purpose, a 3D-scan of a breast of a patient 92 (depicted in FIG. 8B) is obtained using a 3D-scanner. Subsequently, a second 3D-scan is obtained of a mastectomized chest 94 (depicted in FIG. 8B) of a patient. A 3D-model can then be calculated on the first and second 3D-scan. This model can be used to create a front mould model, a rear mould model, and a mould core model. These models can then be used to create the front mould, rear mould, and mould core.

Looking at FIGS. 4A, 4B, and 4C, the front mould and part of its production process is shown. In FIG. 4A, an intermediary mould 26A is shown that corresponds to the front mould model. Such an intermediary mould can be created by additive manufacturing, in particular 3D-printing. To create the front mould 20, a mould material, such as PET, is vacuum formed onto the intermediary front mould 26A (depicted in FIG. 4B), the intermediary front mould being a positive vacuum forming mould. Looking at FIG. 4C, another possible intermediary front mould 26B is shown. Here, the mould material will not be drawn over the intermediary front mould, but into it, the intermediary front mould 26B being a female vacuum forming mould.

If a print resolution of a 3D-printer would be sufficient, it would also be possible to 3D-print a front mould corresponding to the front mould model. This would look similar to the female vacuum forming mould 26B.

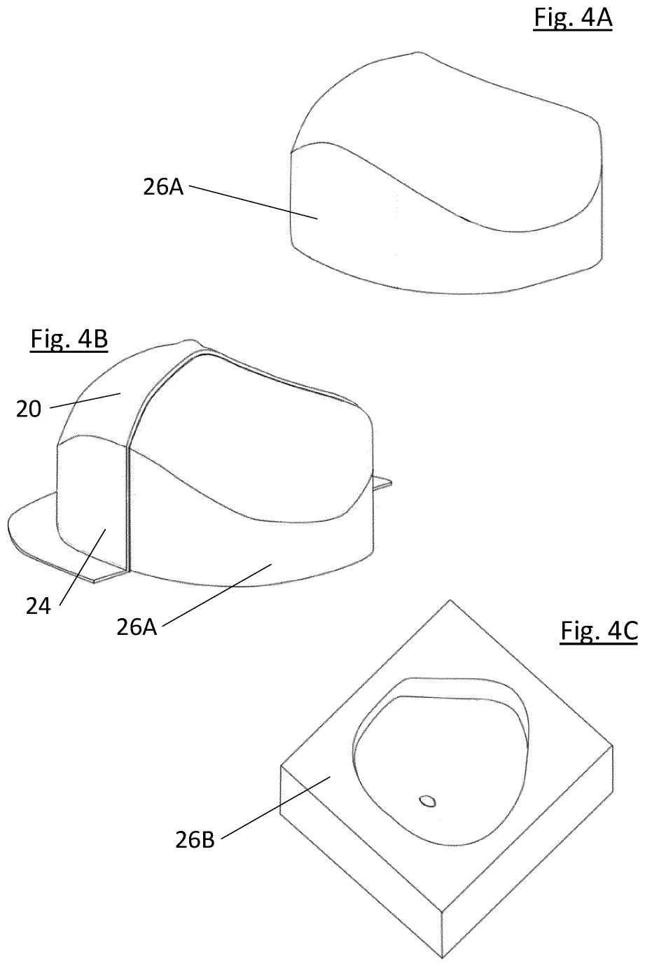

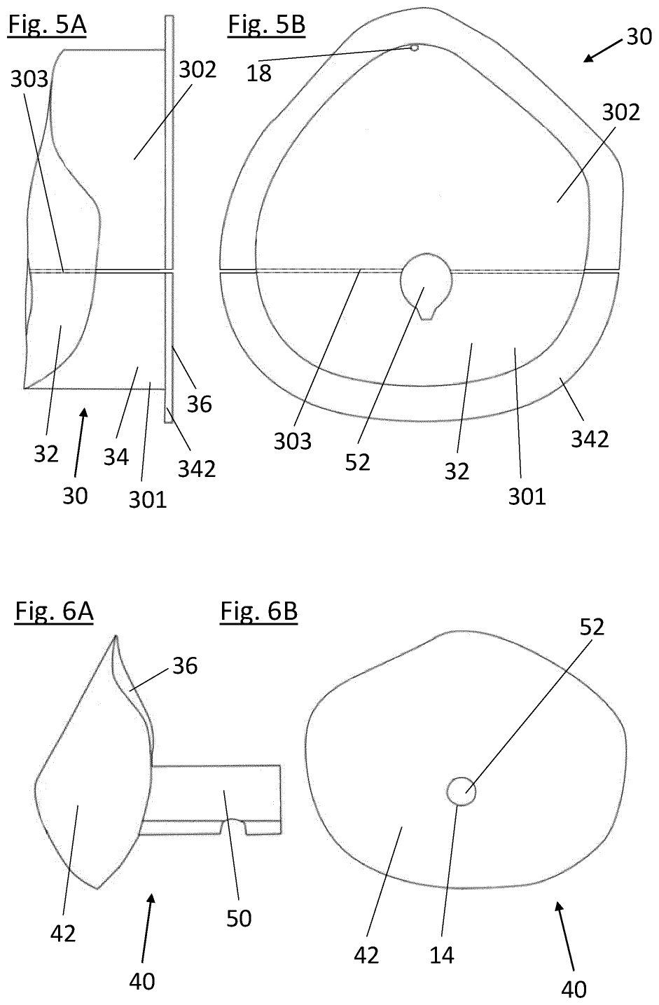

Turning to FIGS. 5A, 5B, 6A, and 6B, the rear mould 30 and the mould core 40 are shown. The rear mould has been manufactured by 3D-printing the rear mould model and the mould core has been manufactured by 3D-printing the mould core model. Because the second mould surface 32 creates the rear side of the prosthesis that will be located on the mastectomized chest of a patient, it is important that any imperfections are reduced. Similarly, because surface imperfections on the mould core surface can unpredictably alter the wall thickness of a prosthesis, imperfections are reduced. This is achieved by covering the moulds with a filler material such as epoxy. To avoid or reduce this step, errors and/or imperfections may also be removed from at least one of the first 3D-scan, the second 3D-scan, the 3D-model of the breast, the 3D-model of the mastectomized chest, the front mould model, the rear mould model, the mould core model by computer editing.

FIG. 5B also shows another embodiment of the air channel 18. Here, instead of being located between the support 50 and the support hole 52, the air channel is a through hole that allows air to escape when the mould assembly is filled.

In FIGS. 5A and 5B, the rear mould 30 comprises a separate first part 301 and a separate second part 302. Both parts are connected to each other by a bridge section 303 that is configured to be broken once a breast prosthesis has been made using the mould assembly. This may facilitate the unmoulding of the breast prosthesis. To this end, the bridge section 303 may be very thin. The bridge section may be 3D-printed together with the separate first part 301 and the separate second part 302 during step e) to form an integral part. However, the separate first part and the separate second part may also be individually 3D-printed and may be joined to each other by melting and solidifying a polymer between them and in doing so creating the bridge section 303. The bridge section 303 is not only present to keep the separate first part 301 and the separate second part 302 together and at a specific position, it is also present to reduce injected polymer from flowing between the separate first part and the separate second part. The dotted lines are present to illustrate the location of the bridge section but are not necessarily present in the 3D-printed rear mould itself.

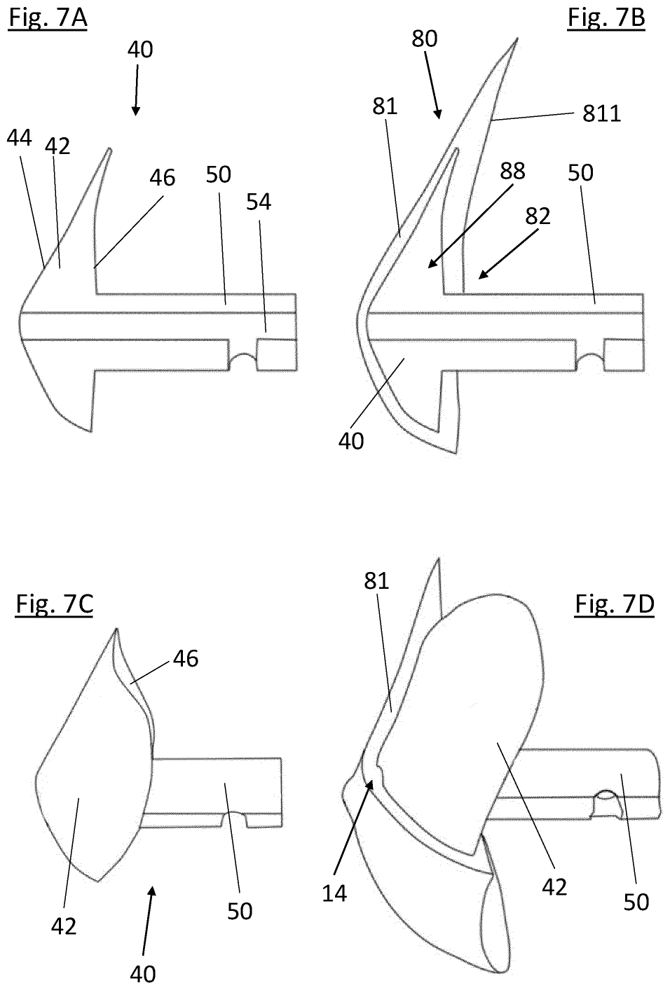

In FIGS. 7A, 7B, 7C, and 7D, the mould core 40 is depicted in side view. In FIGS. 7A and 7C, the mould core 40 is shown standing alone with the support 50 extending away from the rear side 50 of the mould core main body 42. In FIGS. 7B and 7D, the mould core is shown together with a breast prosthesis 80 where a front part and rear part are a single integral part. After having injected a material into the mould assembly, the mould assembly is disassembled by separating the front mould, the rear mould, and the mould core, leaving the mould core 40 with the prosthesis 80.

The material has formed a shell 81 where formerly the inner volume of the mould assembly was and the mould core occupies an inner volume 88 of the breast prosthesis 80. The mould core 40 can then be removed from the breast prosthesis 80 through the hole 82 located in the rear side 811 of the prosthesis. It is necessary to stretch the hole 82 to be able to remove the mould core main body 42. However, because the material is flexible this isn't an issue. Here, the support 50 can be used as a handle to exert a force on the mould core. It is also the support 50 that has created the hole 82 during the injection moulding.

Turning to FIGS. 8A, 8B, 8C, and 8D, a patient's 90 torso is shown. In FIG. 8D, the prosthesis 80 that has been manufactured with the mould assembly has been placed on the mastectomized chest 94 of the patient. If the patient's breast has already been mastectomized prior to the 3D-scanning of the first scan, it is also possible to scan the not to be mastectomized breast and to mirror the scan or the resulting model to manufacture the prosthesis.

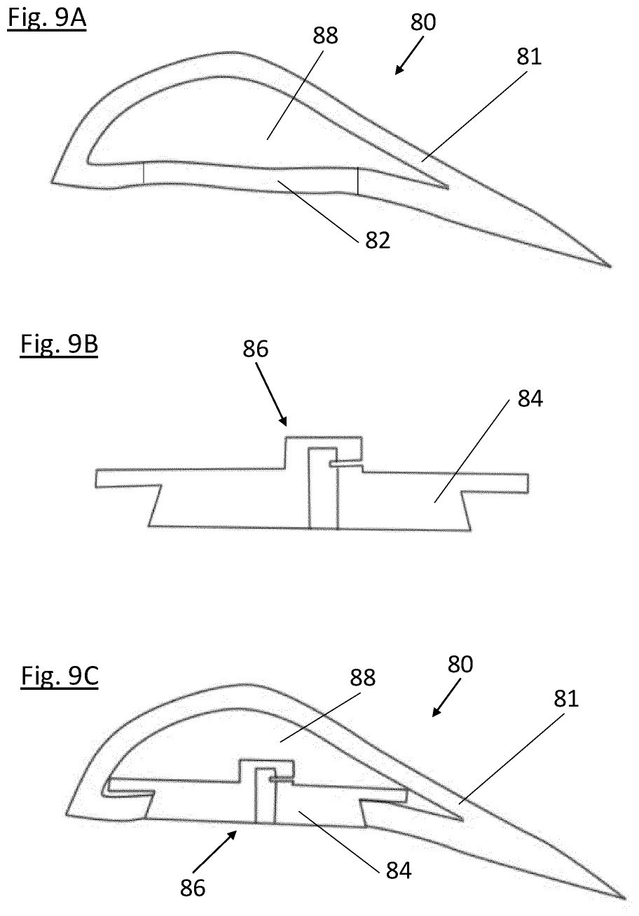

Turning to FIGS. 9A, 9B, and 9C, the hollow breast prosthesis 80 and its further components are depicted. In FIG. 9A, the breast prosthesis 80 is shown just as the mould core has been removed from its inner volume 88. Because the hole 82 is a large and open area, the shell 81 is the only part that provides a resilient structure. To promote a more realistic feeling the hole 82 is closed with a seal 84. Because the inner volume 88 is now closed off, the breast prosthesis becomes much more lifelike. Further, in order to adjust the feeling and possibly the size, the seal 84 comprises a valve 86 through which a medium, such as air, can be inserted or drained.

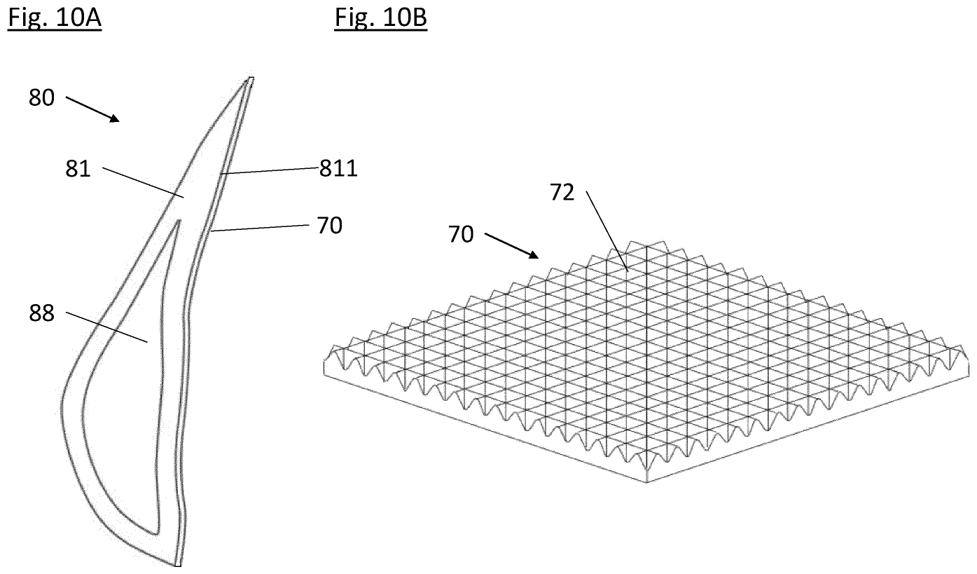

Once the breast prosthesis 80 is to the liking of the patient, an adhering patch 70 is fixed to the rear of the breast prosthesis. This is depicted in FIGS. 10A and 10B. The adhering patch 70 is configured to interact with a patient's skin to adhere to a patient's chest and may comprise a nano-structure 72 that avoids using unpleasant adhesives such as glue. An example of such a nano-structure is a negative of a diffraction grating and may be manufactured by pouring liquid silicone over a diffraction grating and letting it harden. In the case where the injection moulded polymer, i.e. the material of the breast prosthesis, is silicone, the patch 70 can be fixed to the prosthesis with liquid silicone which is then hardened. The material of the seal 84 against which the patch is also located may also be silicone.

The adhering patch may substantially cover the rear of the breast prosthesis and may substantially match a rear surface of the breast prosthesis. Alternatively, a plurality of adhering patches may cover part of the rear of the breast prosthesis, in particular the patches being circular and/or elongated in form.

The terms “a” or “an”, as used herein, are defined as one or more than one. The term plurality, as used herein, is defined as two or more than two. The term another, as used herein, is defined as at least a second or more. The terms including and/or having, as used herein, are defined as comprising i.e., open language, not excluding other elements or steps.

Any reference signs in the claims should not be construed as limiting the scope of the claims or the invention. It will be recognized that a specific embodiment as claimed may not achieve all of the stated objects.

The mere fact that certain measures are recited in mutually different dependent claims does not indicate that a combination of these measures cannot be used to advantage.

White lines between text paragraphs in the text above indicate that the technical features presented in the paragraph may be considered independent from technical features discussed in a preceding paragraph or in a subsequent paragraph.

1.-60. (canceled)

61. A mould assembly for injection moulding a personalised hollow breast prosthesis, wherein the mould assembly comprises:
a front mould being a female mould of a front side of a patients breast and comprising a first mould surface,
a rear mould being a female mould of a patients mastectomized chest and comprising a second mould surface,
a mould core comprising a mould core main body being a scaled positive mould of a combination of the patients breast and the patients mastectomized chest, wherein the mould core is smaller than the combination,
a support configured to support the mould core main body in an assembled state of the mould assembly and to hold the mould core main body in a predetermined position relative to the front mould and rear mould,
wherein an injection opening is defined by any of the front mould, the rear mould, and the mould core, and wherein in the assembled state the mould core is located between the front mould and the rear mould in order for a mould core surface to be located at a distance from the first mould surface and the second mould surface, wherein in the assembled state an inner volume is defined by the front mould, the rear mould and the mould core, wherein the inner volume extends around the mould core and is configured to be filled with an injection moulding material.

62. The mould assembly according to claim 61, wherein the support and the mould core are integral and the support protrudes outwards from a rear side of the mould core main body and is configured to engage the rear mould, wherein the support has a smaller cross-section than the mould core main body.

63. The mould assembly according to claim 62, wherein the rear mould defines a support hole, wherein in the assembled state the support extends into the support hole, in particular the support hole being a through hole wherein the support extends into the support hole and is flush with a rear side of the rear mould.

64. The mould assembly according to claim 61, wherein the support is part of the rear mould and protrudes away from the second mould surface and towards the mould core and is configured to engage the mould core, wherein the second mould surface is adjacent to the inner volume.

65. The mould assembly according to claim 64, wherein the mould core defines a support hole, wherein in the assembled state the support extends into the support hole wherein the support and the support hole have a substantially equal, non-circular cross-section.

66. The mould assembly according to claim 61, wherein the support extends between the mould core and the rear mould and wherein the support, the mould core and the rear mould are an integral part, or
wherein the support extends between the mould core and the front mould and wherein the support, the mould core and the front mould are an integral part.

67. The mould assembly according to claim 61, wherein the injection opening is located at a front side of the inner volume and is configured to allow injection of a polymer near the front mould.

68. The mould assembly according to claim 61, wherein an injection channel extends through the support and through the mould core main body and to the injection opening and is configured to allow injection of a polymer from outside the mould assembly into the mould assembly, in particular the injection channel having a diameter of 3-15 mm, in particular 5-11 mm, more in particular 7-9 mm

69. The mould assembly according to claim 61, wherein the front mould is placed over the rear mould in the assembled state, or vice versa, and wherein at least one of the front mould and the rear mould defines at least one air channel being configured to allow air to escape when the mould assembly is being filled, in particular the at least one air channel having a diameter of 0.5-3 mm, in particular 1-2 mm.

70. The mould assembly according to claim 61, wherein the cross-section of the support is smaller than a cross-section of the support hole and wherein, in the assembled state, an air channel is defined between the support and the support hole.

71. The mould assembly according to claim 61, wherein in the assembled state the front mould is fixed to the rear mould by fixating means, form-fit and/or force-fit, and/or wherein the mould core is fixed to the rear mould by fixating means, form-fit and/or force-fit.

72. The mould assembly according to claim 61, wherein the front mould comprises a skirt and the rear mould comprises a wall, or vice versa, wherein in the assembled state, the skirt is form-fitted to the wall.

73. A method for injection moulding a personalized hollow breast prosthesis using a mould assembly according to claim 61, wherein the method comprises the steps:
a) obtaining a first 3D-scan of a breast of a patient by 3D-scanning the patient,
b) obtaining a second 3D-scan of a mastectomized chest of a patient by 3D-scanning the patient,
c) calculating a 3D-model of the breast based on the first 3D-scan and calculating a 3D-model of the mastectomized chest based on the second 3D-scan,
d) creating a front mould model, a rear mould model, and a mould core model based on the 3D-model of the breast and the 3D-model of the mastectomized chest,
e) 1) 3D-printing an intermediary front mould corresponding to the front mould model and creating a front mould from the intermediary front mould, 3D-printing a rear mould corresponding to the rear mould model, and 3D-printing a mould core corresponding to the mould core model, or
2) 3D-printing a front mould corresponding to the front mould model, 3D-printing a rear mould corresponding to the rear mould model, and 3D-printing a mould core corresponding to the mould core model,
f) assembling the front mould, the rear mould, and the mould core into the mould assembly,
g) injecting a polymer into the mould assembly through an injection opening creating the hollow breast prosthesis,
wherein in the assembled state a mould core main body is supported by a support to position a mould core surface at a distance from the front mould and the rear mould.

74. The method according to claim 73, wherein in step e1) the front mould is created by vacuum forming a mould material onto the intermediary front mould, in particular the mould material being PET.

75. The method according to claim 73, wherein after step e) at least one of the front mould, the intermediary front mould, the rear mould, and the mould core are covered with a filler material to reduce surface imperfections, in particular the filler material being epoxy.

76. The method according to claim 73, wherein the mould core defines an injection channel extending to the injection opening and wherein during step g) the polymer flows through the injection channel of the mould core into the mould assembly.

77. The method according to claim 73, wherein the rear mould comprises a separate first part, a separate second part, and a bridge section connecting the separate first part and the separate second part that is configured to be broken after step g), and wherein during step e) the separate first part, the separate second part and the bridge section are 3D-printed into an integral part.

78. The method according to claim 73, wherein a hole in the hollow breast prosthesis is closed with a seal after removing the mould core.

79. The method according to claim 73, wherein the hollow breast prosthesis comprises a valve and the hollow breast prosthesis is filled with a medium or is drained of a medium through the valve, in particular the valve being located in the seal, more in particular the medium being air.

80. The method according to claim 73, wherein the first 3D-scan is obtained from a breast that is not to be mastectomized and wherein prior to step d) the first 3D-scan or the 3D-model of the breast based on the first 3D-scan is mirrored.
